(19) United States
(12) Patent Application Publication
(10) Pub. No.: US 2022/0157416 A1
(43) Pub. Date: May 19, 2022

(54) DISEASE PROGRESSION HAZARD RATIO BASED ON EHR DATABASE

(71) Applicant: International Business Machines Corporation, Armonk, NY (US)

(72) Inventors: Ze Fang Tang, Beijing (CN); Bu Yu Gao, Yinchuan (CN); Yuan Zhou, Beijing (CN)

(21) Appl. No.: 16/951,245

(22) Filed: Nov. 18, 2020

(51) Int. Cl. G16H 10/60; G16H 50/20; G06F 17/18

(52) U.S. Cl. CPC G16H 10/60; G06F 17/18; G16H 50/20

(57) ABSTRACT

In an approach for identifying disease progression hazard ratios for given disease against diseases from an EHR database to determine top comorbidities to the given disease, a processor receives raw EHR data. A processor identifies, from the raw EHR data, a set of diseases and associated diagnosis information for each disease of the set of diseases. A processor calculates a hazard ratio for each disease pair of a set of disease pairs producing a set of hazard ratios, wherein the set of disease pairs comprises a given disease paired with each disease of the set of diseases. A processor ranks the set of hazard ratios for the given disease. A processor selects a pre-defined number of top comorbidities of the set of hazard ratios for the given disease based on the ranking. A processor outputs the pre-defined number of top comorbidities for the given disease.

### BACKGROUND OF THE INVENTION

The present invention relates generally to the field of disease progression, and more particularly to identifying disease progression hazard ratios for disease pairs based on Electronic Health Records database.

An electronic health record (EHR) is a digital version of a patient's paper chart. EHRs are real-time, patient-centered records that make information available instantly and securely to authorized users. While an EHR does contain the medical and treatment histories of patients, an EHR system is built to go beyond standard clinical data collected in a provider's office and can be inclusive of a broader view of a patient's care. EHRs are a vital part of health information technology and can contain a patient's medical history, diagnoses, medications, treatment plans, immunization dates, allergies, radiology images, and laboratory and test results; allow access to evidence-based tools that providers can use to make decisions about a patient's care; and automate and streamline provider workflow.

One of the key features of an EHR is that health information can be created and managed by authorized providers in a digital format capable of being shared with other providers across more than one health care organization. EHRs are built to share information with other health care providers and organizations—such as laboratories, specialists, medical imaging facilities, pharmacies, emergency facilities, and school and workplace clinics—so they contain information from all clinicians involved in a patient's care.

### SUMMARY

Aspects of an embodiment of the present invention disclose a method, computer program product, and computer system for identifying disease progression hazard ratios for given disease against diseases from an EHR database to determine top comorbidities to the given disease. A processor receives raw EHR data. A processor identifies, from the raw EHR data, a set of diseases and associated diagnosis information for each disease of the set of diseases. A processor calculates a hazard ratio for each disease pair of a set of disease pairs producing a set of hazard ratios, wherein the set of disease pairs comprises a given disease paired with each disease of the set of diseases. A processor ranks the set of hazard ratios for the given disease. A processor selects a pre-defined number of top comorbidities of the set of hazard ratios for the given disease based on the rankings. A processor outputs the pre-defined number of top comorbidities for the given disease.

In some aspects of an embodiment of the present invention, the raw EHR data is received from one EHR database.

In some aspects of an embodiment of the present invention, the associated diagnosis information comprises a diagnosis time when a person was diagnosed with a disease.

In some aspects of an embodiment of the present invention, a processor calculates the respective hazard ratio (HR) for a disease pair of the set of disease pairs using

$$H {R}_{i , j , l} = \frac{{I}_{o b s e r v e d}}{{I}_{r a n d o m}} = \frac{{I}_{i , j , l}}{{\sum }_{k = 1}^{{n}_{b}} I {.}_{, j , l}^{k} / {n}_{b}} , ∀ i , j ∈ D ,$$

wherein D is a disease space of the EHR raw data, I_{observed} is a percentage of patients who get disease j after diagnosis of disease i within l-year interval, I_{random} is a mean value of randomly sampled incidence

$$I {.}_{, j , l}^{k}$$

of disease j within the l-year interval using n_{b} times bootstrap.

In some aspects of an embodiment of the present invention, the pre-defined number of top comorbidities is pre-defined by a user through a computing device.

In some aspects of an embodiment of the present invention, a processor generates a visualization of each disease of the set of diseases that connects to another disease of the set of diseases based on the hazard ratio for each disease pair. A processor visually outputs the visualization to a user through a user interface of a computing device.

In some aspects of an embodiment of the present invention, a processor outputs the pre-defined number of top comorbidities for the given disease to a user through a user interface of a computing device.

### BRIEF DESCRIPTION OF THE DRAWINGS

FIG. 1 is a functional block diagram illustrating a distributed data processing environment, in accordance with an embodiment of the present invention.

FIG. 2 is a flowchart depicting operational steps of the hazard ratio program, for identifying disease progression hazard ratios for given disease against diseases from an EHR database to determine top comorbidities to the given disease, in accordance with an embodiment of the present invention.

FIG. 3 depicts a block diagram of components of a computing device of the distributed data processing environment of FIG. 1, in accordance with an embodiment of the present invention.

### DETAILED DESCRIPTION

Embodiments of the present invention recognize that, in the last 10 years, EHR data has become popular in artificial intelligence applications for the healthcare field. Researchers and doctors are interested in the connections between diseases, especially the progression pathway of a given disease that could be further used for disease risk modelling. For example, given disease A and disease B, how to calculate the relative hazard ratio of disease B after the diagnosis of disease A is a valuable and challenging issue. Thus, there is a need for a solution that can calculate the relative hazard ratio of disease B after the diagnosis of disease A.

Embodiments of the present invention provide a solution for identifying a disease progression hazard ratio for any given disease pair based on an EHR database. Thus, embodiments of the present invention can mine and quantify more implicit dependencies and relations between each disease, which cannot be observed from single or partial disease information. Once a patient has a first disease, doctors could use this solution to prevent later diseases with high hazard ratio values showing a strong relationship or connection to the first disease. Embodiments of the present invention provide a method and system for identifying disease progression hazard ratios for given disease against diseases from an EHR database to determine top comorbidities to the given disease.

Embodiments of the present invention provide a program that automatically produces the hazard ratio for a given disease pair using an explainable algorithm that dynamically calculates the hazard ratio, which could be applied in any EHR database. Embodiments of the present invention further provide a program that takes the calculated hazard ratios and presents the hazard ratios visually to show corresponding diseases for better explainability. Thus, embodiments of the present invention do not require corresponding disease information and do not need to build deep neural networks based on large datasets and prior knowledge, and thus, are less time consuming.

Implementation of embodiments of the invention may take a variety of forms, and exemplary implementation details are discussed subsequently with reference to the Figures.

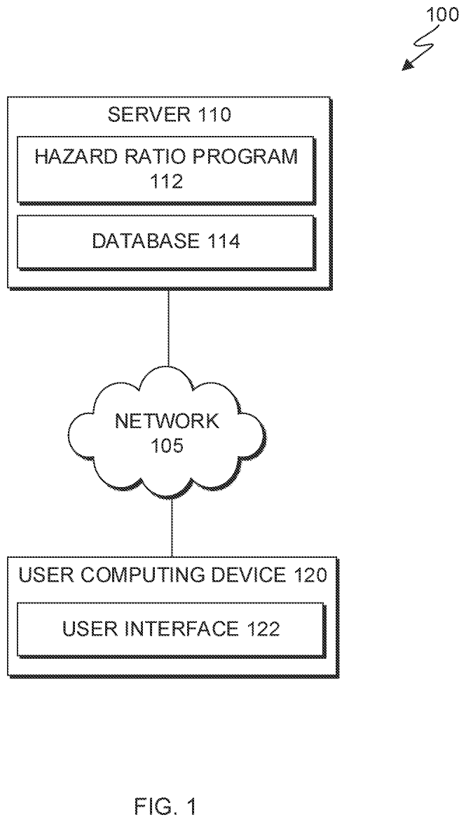

FIG. 1 is a functional block diagram illustrating a distributed data processing environment, generally designated 100, in accordance with one embodiment of the present invention. The term “distributed,” as used herein, describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Distributed data processing environment 100 includes server 110 and user computing device 120, interconnected over network 105. Network 105 can be, for example, a telecommunications network, a local area network (LAN), a wide area network (WAN), such as the Internet, or a combination of the three, and can include wired, wireless, or fiber optic connections. Network 105 can include one or more wired and/or wireless networks capable of receiving and transmitting data, voice, and/or video signals, including multimedia signals that include voice, data, and video information. In general, network 105 can be any combination of connections and protocols that will support communications between server 110, user computing device 120, and other computing devices (not shown) within distributed data processing environment 100.

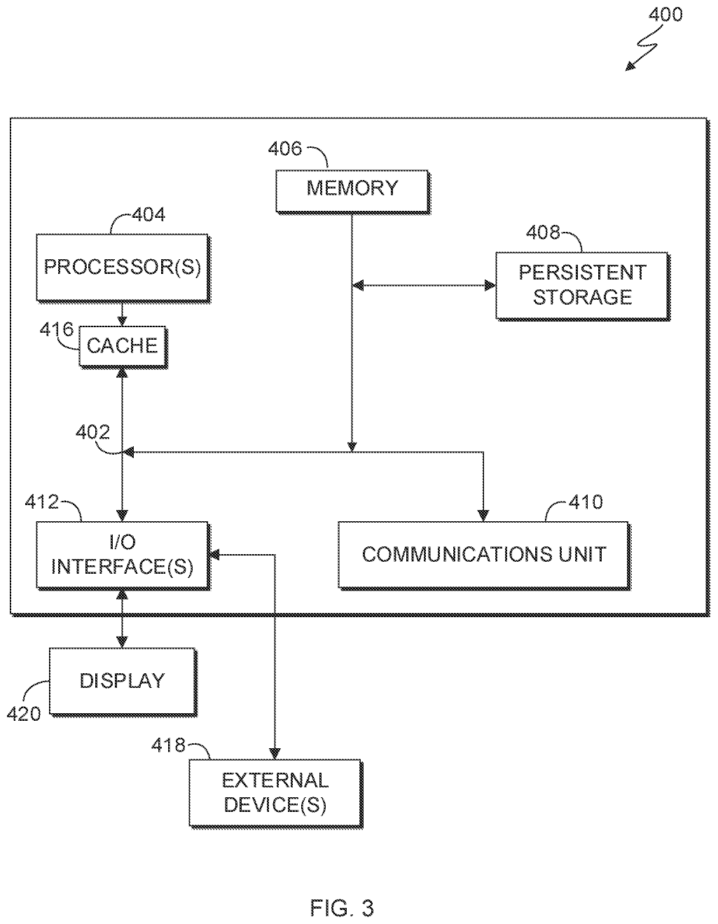

Server 110 can be a standalone computing device, a management server, a web server, a mobile computing device, or any other electronic device or computing system capable of receiving, sending, and processing data. In other embodiments, server 110 can represent a server computing system utilizing multiple computers as a server system, such as in a cloud computing environment. In another embodiment, server 110 can be a laptop computer, a tablet computer, a netbook computer, a personal computer (PC), a desktop computer, a personal digital assistant (PDA), a smart phone, or any programmable electronic device capable of communicating with user computing device 120, and other computing devices (not shown) within distributed data processing environment 100 via network 105. In another embodiment, server 110 represents a computing system utilizing clustered computers and components (e.g., database server computers, application server computers, etc.) that act as a single pool of seamless resources when accessed within distributed data processing environment 100. Server 110 includes hazard ratio program 112 and database 114. Server 110 may include internal and external hardware components, as depicted and described in further detail with respect to FIG. 3.

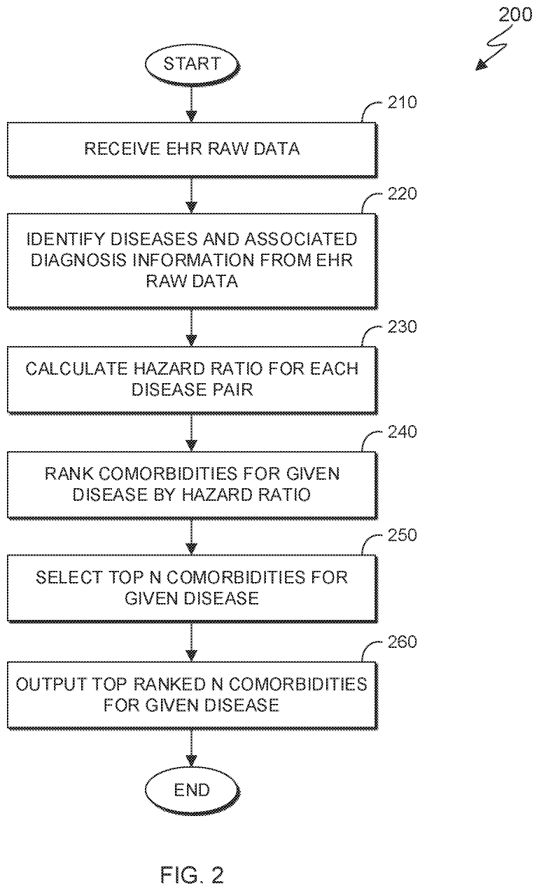

Hazard ratio program 112 operates to identify disease progression hazard ratios for a given disease against diseases from an EHR database to determine top comorbidities to the given disease. In the depicted embodiment, hazard ratio program 112 is a standalone program. In another embodiment hazard ratio program 112 may be integrated into another software product, such as a medical research software package. Hazard ratio program 112 is depicted and described in further detail with respect to FIG. 2.

Database 114 operates as a repository for data received, used, and/or output by hazard ratio program 112. Data received, used, and/or generated may include, but is not limited to, raw EHR data; and any other data received, used, and/or output by hazard ratio program 112. Database 114 can be implemented with any type of storage device capable of storing data and configuration files that can be accessed and utilized by server 110, such as a hard disk drive, a database server, or a flash memory. In an embodiment, database 114 is accessed by hazard ratio program 112 to store and/or to access the data. In the depicted embodiment, database 114 resides on server 110. In another embodiment, database 114 may reside on another computing device, server, cloud server, or spread across multiple devices elsewhere (not shown) within distributed data processing environment 100, provided that hazard ratio program 112 has access to database 114.

The present invention may contain various accessible data sources, such as database 114, that may include personal and/or confidential company data, content, or information the user wishes not to be processed. Processing refers to any operation, automated or unautomated, or set of operations such as collecting, recording, organizing, structuring, storing, adapting, altering, retrieving, consulting, using, disclosing by transmission, dissemination, or otherwise making available, combining, restricting, erasing, or destroying personal and/or confidential company data. Hazard ratio program 112 enables the authorized and secure processing of personal data.

Hazard ratio program 112 provides informed consent, with notice of the collection of personal and/or confidential company data, allowing the user to opt in or opt out of processing personal and/or confidential company data. Consent can take several forms. Opt-in consent can impose on the user to take an affirmative action before personal and/or confidential company data is processed. Alternatively, opt-out consent can impose on the user to take an affirmative action to prevent the processing of personal and/or confidential company data before personal and/or confidential company data is processed. Hazard ratio program 112 provides information regarding personal and/or confidential company data and the nature (e.g., type, scope, purpose, duration, etc.) of the processing. Hazard ratio program 112 provides the user with copies of stored personal and/or confidential company data. Hazard ratio program 112 allows the correction or completion of incorrect or incomplete personal and/or confidential company data. Hazard ratio program 112 allows for the immediate deletion of personal and/or confidential company data.

User computing device 120 operates as a computing device associated with a user on which the user can view a final output from hazard ratio program 112 through an application user interface. In the depicted embodiment, user computing device 120 includes an instance of user interface 122. In an embodiment, user computing device 120 can be a laptop computer, a tablet computer, a smart phone, a smart watch, an e-reader, smart glasses, wearable computer, or any programmable electronic device capable of communicating with various components and devices within distributed data processing environment 100, via network 105. In general, user computing device 120 represents one or more programmable electronic devices or combination of programmable electronic devices capable of executing machine readable program instructions and communicating with other computing devices (not shown) within distributed data processing environment 100 via a network, such as network 105. User computing device 120 may include internal and external hardware components, as depicted and described in further detail with respect to FIG. 4.

User interface 122 provides an interface between hazard ratio program 112 on server 110 and a user of user computing device 120. In one embodiment, user interface 122 is a mobile application software. Mobile application software, or an “app,” is a computer program designed to run on smart phones, tablet computers, and other mobile computing devices. In one embodiment, user interface 122 may be a graphical user interface (GUI) or a web user interface (WUI) that can display text, documents, web browser windows, user options, application interfaces, and instructions for operation, and include the information (such as graphic, text, and sound) that a program presents to a user and the control sequences the user employs to control the program. User interface 122 enables a user of user computing device 120 to view a final output from hazard ratio program 112.

FIG. 2 is a flowchart 200 depicting operational steps of hazard ratio program 112, for identifying disease progression hazard ratios for a given disease against diseases from an EHR database to determine top comorbidities to the given disease, in accordance with an embodiment of the present invention. It should be appreciated that the process depicted in FIG. 2 illustrates one possible iteration of hazard ratio program 112, which may be initiated upon hazard ratio program 112 receiving a given disease. For example, a user inputting a given disease, e.g., through user interface 122 of user computing device 120, for hazard ratio program 112 to determine the top hazard ratios for all diseases in an EHR database in relation to the given disease, and thus, identifying the top comorbidities to the given disease.

In step 210, hazard ratio program 112 receives EHR raw data. EHR raw data is considered “raw” because the data has been input by a user and not processed. In an embodiment, hazard ratio program 112 receives EHR raw data from one EHR database. The raw data can come from any EHR database, such as the Explorys database, Truven database, or the Unified Data Model for Healthcare (UDMH) system, but only the raw data from one EHR database is used at a time. In some embodiments, hazard ratio program 112 receives the EHR raw data from a database, e.g., database 114. In other embodiments, hazard ratio program 112 receives the EHR raw data from a computing device, e.g., user computing device 120.

In step 220, hazard ratio program 112 identifies diseases and associated diagnosis information from the EHR raw data. In an embodiment, hazard ratio program 112 identifies every disease and associated diagnosis information for each disease from the EHR raw data. In an embodiment, responsive to hazard ratio program 112 receiving the raw EHR data, hazard ratio program 112 identifies diseases and associated diagnosis information from the EHR raw data. The associated diagnosis information includes a diagnosis time, i.e., when a person was diagnosed with the disease.

In step 230, hazard ratio program 112 calculates hazard ratio for each disease pair. In an embodiment, responsive to identifying the diseases and associated diagnosis information, hazard ratio program 112 calculates a hazard ratio for each disease pair. A disease pair represents a given disease paired with a disease identified from the EHR raw data. Thus, hazard ratio program 112 pairs each disease identified from the EHR raw data with a given disease to create a disease pair and calculate a hazard ratio for. In an embodiment, hazard ratio program 112 analyzes a progression pathway to identify comorbidities of a given disease using an incidence odds ratio (OR) algorithm. A disease pair with a higher hazard ratio indicates a stronger relationship between the two diseases, and thus, the hazard ratio reflects the connection between the two diseases. For a disease of interest i (i.e., the given disease), hazard ratio program 112 identifies comorbidities of disease i among other diseases based on their incidence OR. Hazard ratio program 112 calculates a hazard ratio of each disease j after the diagnosis of disease i. The incidence OR of disease j towards disease i is represented by equation (1) below.

$${OR}_{i , j , l} = \frac{{I}_{o b s e r v e d}}{{I}_{r a n d o m}} = \frac{{I}_{i , j , l}}{{\sum }_{k = 1}^{{n}_{b}} I {.}_{, j , l}^{k} / {n}_{b}} , ∀ i , j ∈ D \qquad ( 1 )$$

In equation (1), D is a disease space of the EHR database, I_{observed} is a percentage of patients who get disease j after diagnosis of disease i within l-year interval, I_{random} is a mean value of randomly sampled incidence

$$I {.}_{, j , l}^{k}$$

of disease j within the l-year interval using n_{b} times bootstrap. The incident OR values represent the hazard ratios (HR) of a disease j incidence after disease i is diagnosed.

In step 240, hazard ratio program 112 ranks comorbidities for given disease by hazard ratio. In an embodiment, responsive calculating a hazard ratio for each disease pair, hazard ratio program 112 ranks comorbidities for given disease by hazard ratio. In an embodiment, hazard ratio program 112 ranks the hazard ratios highest to lowest, in which higher hazard ratios reflect a stronger connection between the two diseases of the disease pair. In each disease pair, the disease paired with the given disease would be considered a comorbidity of the given disease.

In step 250, hazard ratio program 112 selects top N comorbidities for the given disease. In an embodiment, responsive to ranking comorbidities for given disease by hazard ratio, hazard ratio program 112 selects the top N comorbidities for the given disease. The value N is any positive integer that can be pre-defined by a user, e.g., through user interface 122 of user computing device 120.

In step 260, hazard ratio program 112 outputs top ranked N comorbidities for given disease. In an embodiment, hazard ratio program 112 outputs the top ranked N comorbidities with associated hazard score for the given disease. In an embodiment, hazard ratio program 112 outputs a visualization of the diseases and how they connect (i.e., have a relationship) to another disease based on their hazard ratio. In an embodiment, hazard ratio program 112 outputs the top ranked N comorbidities with associated hazard score for the given disease and/or visualization to a user through a user interface of a user computing device. In an embodiment, responsive selecting the top N comorbidities for a given disease, hazard ratio program 112 outputs the top ranked N comorbidities for the given disease.

FIG. 3 depicts a block diagram of components of computing device 400, suitable for server 110 and/or user computing device 120 within distributed data processing environment 100 of FIG. 1, in accordance with an embodiment of the present invention. It should be appreciated that FIG. 3 provides only an illustration of one implementation and does not imply any limitations with regard to the environments in which different embodiments can be implemented. Many modifications to the depicted environment can be made.

Computing device 400 includes communications fabric 402, which provides communications between cache 416, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a crossbar switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

Programs may be stored in persistent storage 408 and in memory 406 for execution and/or access by one or more of the respective computer processors 404 via cache 416. In an embodiment, persistent storage 408 includes a magnetic hard disk drive. Alternatively, or in addition to a magnetic hard disk drive, persistent storage 408 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices. In these examples, communications unit 410 includes one or more network interface cards. Communications unit 410 may provide communications through the use of either or both physical and wireless communications links. Programs may be downloaded to persistent storage 408 through communications unit 410.

I/O interface(s) 412 allows for input and output of data with other devices that may be connected to server 110 and/or user computing device 120. For example, I/O interface 412 may provide a connection to external devices 418 such as a keyboard, keypad, a touch screen, and/or some other suitable input device. External devices 418 can also include portable computer readable storage media such as, for example, thumb drives, portable optical or magnetic disks, and memory cards. Software and data used to practice embodiments of the present invention can be stored on such portable computer readable storage media and can be loaded onto persistent storage 408 via I/O interface(s) 412. I/O interface(s) 412 also connect to a display 420.

Display 420 provides a mechanism to display data to a user and may be, for example, a computer monitor.

Programs described herein is identified based upon the application for which it is implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method comprising:
receiving, by one or more processors, raw EHR data;
identifying, by the one or more processors, from the raw EHR data, a set of diseases and associated diagnosis information for each disease of the set of diseases;
calculating, by the one or more processors, a hazard ratio for each disease pair of a set of disease pairs producing a set of hazard ratios, wherein the set of disease pairs comprises a given disease paired with each disease of the set of diseases;
ranking, by the one or more processors, the set of hazard ratios for the given disease;
selecting, by the one or more processors, a pre-defined number of top comorbidities of the set of hazard ratios for the given disease based on the ranking; and
outputting, by the one or more processors, the pre-defined number of top comorbidities for the given disease.

2. The computer-implemented method of claim 1, wherein the raw EHR data is received from one EHR database.

3. The computer-implemented method of claim 1, wherein the associated diagnosis information comprises a diagnosis time when a person was diagnosed with a disease.

4. The computer-implemented method of claim 1, wherein calculating the hazard ratio for each disease pair of the set of disease pairs producing the set of hazard ratios comprises:
calculating, by the one or more processors, the respective hazard ratio (HR) for a disease pair of the set of disease pairs using
$$H {R}_{i , j , l} = \frac{{I}_{o b s e r v e d}}{{I}_{r a n d o m}} = \frac{{I}_{i , j , l}}{{\sum }_{k = 1}^{{n}_{b}} I {.}_{, j , l}^{k} / {n}_{b}} , ∀ i , j ∈ D ,$$
wherein D is a disease space of the EHR raw data, I_{observed} is a percentage of patients who get disease j after diagnosis of disease i within l-year interval, I_{random} is a mean value of randomly sampled incidence
$$I {.}_{, j , l}^{k}$$
of disease j within the l-year interval using n_{b} times bootstrap.

5. The computer-implemented method of claim 1, where the pre-defined number of top comorbidities is pre-defined by a user through a computing device.

6. The computer-implemented method of claim 1, further comprising:
generating, by the one or more processors, a visualization of each disease of the set of diseases that connects to another disease of the set of diseases based on the hazard ratio for each disease pair; and
visually outputting, by the one or more processors, the visualization to a user through a user interface of a computing device.

7. The computer-implemented method of claim 1, wherein outputting the pre-defined number of top comorbidities for the given disease comprises:
outputting, by the one or more processors, the pre-defined number of top comorbidities for the given disease to a user through a user interface of a computing device.

8. A computer program product comprising:
one or more computer readable storage media and program instructions collectively stored on the one or more computer readable storage media, the stored program instructions comprising:
program instructions to receive raw EHR data;
program instructions to identify, from the raw EHR data, a set of diseases and associated diagnosis information for each disease of the set of diseases;
program instructions to calculate a hazard ratio for each disease pair of a set of disease pairs producing a set of hazard ratios, wherein the set of disease pairs comprises a given disease paired with each disease of the set of diseases;
program instructions to rank the set of hazard ratios for the given disease;
program instructions to select a pre-defined number of top comorbidities of the set of hazard ratios for the given disease based on the ranking; and
program instructions to output the pre-defined number of top comorbidities for the given disease.

9. The computer program product of claim 8, wherein the raw EHR data is received from one EHR database.

10. The computer program product of claim 8, wherein the associated diagnosis information comprises a diagnosis time when a person was diagnosed with a disease.

11. The computer program product of claim 8, wherein the program instructions to calculate the hazard ratio for each disease pair of the set of disease pairs producing the set of hazard ratios comprise:
program instructions to calculate the respective hazard ratio (HR) for a disease pair of the set of disease pairs using
$$H {R}_{i , j , l} = \frac{{I}_{o b s e r v e d}}{{I}_{r a n d o m}} = \frac{{I}_{i , j , l}}{{\sum }_{k = 1}^{{n}_{b}} I {.}_{, j , l}^{k} / {n}_{b}} , ∀ i , j ∈ D ,$$
wherein D is a disease space of the EHR raw data, I_{observed} is a percentage of patients who get disease j after diagnosis of disease i within l-year interval, I_{random} is a mean value of randomly sampled incidence
$$I {.}_{, j , l}^{k}$$
of disease j within the l-year interval using n_{b} times bootstrap.

12. The computer program product of claim 8, where the pre-defined number of top comorbidities is pre-defined by a user through a computing device.

13. The computer program product of claim 8, further comprising:
program instructions to generate a visualization of each disease of the set of diseases that connects to another disease of the set of diseases based on the hazard ratio for each disease pair; and
program instructions to visually output the visualization to a user through a user interface of a computing device.

14. The computer program product of claim 8, wherein the program instructions to output the pre-defined number of top comorbidities for the given disease comprise:
program instructions to output the pre-defined number of top comorbidities for the given disease to a user through a user interface of a computing device.

15. A computer system comprising:
one or more computer processors;
one or more computer readable storage media;
program instructions collectively stored on the one or more computer readable storage media for execution by at least one of the one or more computer processors, the stored program instructions comprising:
program instructions to receive raw EHR data;
program instructions to identify, from the raw EHR data, a set of diseases and associated diagnosis information for each disease of the set of diseases;
program instructions to calculate a hazard ratio for each disease pair of a set of disease pairs producing a set of hazard ratios, wherein the set of disease pairs comprises a given disease paired with each disease of the set of diseases;
program instructions to rank the set of hazard ratios for the given disease;
program instructions to select a pre-defined number of top comorbidities of the set of hazard ratios for the given disease based on the ranking; and
program instructions to output the pre-defined number of top comorbidities for the given disease.

16. The computer system of claim 15, wherein the raw EHR data is received from one EHR database.

17. The computer system of claim 15, wherein the associated diagnosis information comprises a diagnosis time when a person was diagnosed with a disease.

18. The computer system of claim 15, wherein the program instructions to calculate the hazard ratio for each disease pair of the set of disease pairs producing the set of hazard ratios comprise:
program instructions to calculate the respective hazard ratio (HR) for a disease pair of the set of disease pairs using
$$H {R}_{i , j , l} = \frac{{I}_{o b s e r v e d}}{{I}_{r a n d o m}} = \frac{{I}_{i , j , l}}{{\sum }_{k = 1}^{{n}_{b}} I {.}_{, j , l}^{k} / {n}_{b}} , ∀ i , j ∈ D ,$$
wherein D is a disease space of the EHR raw data, I_{observed} is a percentage of patients who get disease j after diagnosis of disease i within l-year interval, I_{random} is a mean value of randomly sampled incidence
$$I {.}_{, j , l}^{k}$$
of disease j within the l-year interval using n_{b} times bootstrap.

19. The computer system of claim 15, further comprising:
program instructions to generate a visualization of each disease of the set of diseases that connects to another disease of the set of diseases based on the hazard ratio for each disease pair; and
program instructions to visually output the visualization to a user through a user interface of a computing device.

20. The computer system of claim 15, wherein the program instructions to output the pre-defined number of top comorbidities for the given disease comprise:
program instructions to output the pre-defined number of top comorbidities for the given disease to a user through a user interface of a computing device.
